(19) United States
(12) Patent Application Publication
(10) Pub. No.: US 2002/0197610 A1
(43) Pub. Date: Dec. 26, 2002

(54) Method and system of identifying biologically active molecules

(76) Inventors: Frank Schmitt, Muenchen (DE); Bernhard Schirm, Gauting (DE); Bernd Kramer, Aachen (DE); Daniel Vitt, Germering (DE); Knut Baumann, Wuerzburg (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 09/885,893

(22) Filed: Jun. 20, 2001

(51) Int. Cl. C12Q 1/68; G06G 7/48; G06G 7/58; G06F 19/00; G01N 33/48; G01N 33/50

(52) U.S. Cl. 435/6; 702/19; 703/11

(57) ABSTRACT

The present invention relates to a method and a system of identifying biologically active molecules. Evaluating receptor or target suitability of molecules is an important task in pharmaceutical drug research. With the increasing employment of automation techniques over the last years within Drug Discovery processes, methods like High-Throughput-Screening (HTS) and High-Throughput-Synthesis have become industry standards in pharmaceutical research. Nowadays, it is possible to test more than 20,000 molecules per day for their biological activities in certain disease targets. Also in the area of chemical synthesis, combinatorial chemistry in combination with automation processes, hundreds of molecules per day can be made physically available. Since based on today's chemical knowledge, more than 10¹⁰⁰ molecules could theoretically be synthesized and tested and several hundreds of thousands molecules are commercially available, computer assisted methods have been developed to select subsets of molecules which are actually supposed to be tested based on their predicted potential of biological activity for certain disease targets.

The present invention relates to a method and a system of identifying biologically active molecules.

Evaluating receptor or target suitability of molecules is an important task in pharmaceutical drug research. With the increasing employment of automation techniques over the last years within Drug Discovery processes, methods like High-Throughput-Screening (HTS) and High-Throughput-Synthesis have become industry standards in pharmaceutical research. Nowadays, it is possible to test more than 20,000 molecules per day for their biological activities in certain disease targets. Also in the area of chemical synthesis, combinatorial chemistry in combination with automation processes, hundreds of molecules per day can be made physically available. Since based on today's chemical knowledge, more than 10100 molecules could theoretically be synthesized and tested and several hundreds of thousands molecules are commercially available, computer assisted methods have been developed to select subsets of molecules which are actually supposed to be tested based on their predicted potential of biological activity for certain disease targets.

Two categories of computer assisted methods serve the purpose of discovering (selecting and/or prioritizing) molecules from data sets of theoretically available molecules for biological activity testing. The first category comprises diversity or similarity based discovery methods, whereas the second category comprises structure based discovery methods. Among the second category, there are database search techniques, as well as (Q)SAR methods and Docking methods.

Only the (Q)SAR methods and the Docking methods implicitly consider information related to specific targets, either common structural patterns of a series of active molecules ((Q)SAR) or the 3-dimensional structure of a target protein (Docking) and therefore deliver the most specific results. In practice, methods based on (Q)SAR or Docking are applied to smaller data sets (up to 50,000 sets), since they need relatively high computing power. However, although parallel computing techniques can be used to gain speed, still data sets consisting of more than 106 molecules are not predictable with respect to their biological activity in a reasonable time frame.

The term biological activity is hereinafter used to comprise in particular pharmaceutical as well as agrochemical activity with respect to a certain receptor or target.

The search for candidate molecules also comprises the search for lead compounds.

It is therefore an object of the present invention to provide a method of and a system for finding candidate molecules expected to be biologically active, which method and system can be applied on molecule libraries comprising high amounts of data and yields results in a reasonable time.

This object is achieved by the method and the system according to the independent claims. Advantageous embodiments are defined in the dependent claims.

According to the invention provided is a method of identifying biologically active molecules from a set S comprising a predetermined number N of different molecules M1, M2, . . . , MN, said molecules being expected to be biologically active with respect to a predetermined target T, each said molecule M1, M2, . . . , MN of said set S being identified by a machine-readable descriptor X1, X2, . . . , XN, respectively, each said descriptor X1, . . . , XN being a vector with n vector elements x1, . . . , xn, n being a natural number, each vector element x1, . . . , xn representing a predetermined molecular property, said method comprising the following steps:

a) selecting arbitrarily from said set S of molecules a subset Su comprising a predetermined first number Nu of molecules Mi, . . . , Mk;

b) assigning a fitness fi, . . . , fk, to each molecule Mi, . . . , Mk of said subset Su, respectively, said fitness fi, . . . , fk being calculated according to a predetermined fitness measure f(X), said fitness measure f(X) being representative of the affinity of a molecule Mi, . . . , Mk to said target T;

e) establishing, according to a predetermined selection criterion SC, from said subset Su a predetermined number nc of couples of molecules MX, MY;

f) with each established couple of molecules: producing a predetermined number of descendant molecules MO1, MO2 by recombining the descriptors X, Y of said couple of molecules MX, MY according to a predetermined recombination scheme;

g) mutating each said descendant molecule XM by modifying the respective descriptor XO according to a predetermined mutation scheme MS;

h) assigning a fitness f to each modified descendant molecule MO, said fitness f(MO) being calculated according to the fitness measure f(X) of step b);

i) adding said modified molecules MO to said subset Su;

j) removing a predetermined number of molecules from said subset Su, the molecules to be removed being determined by a predetermined removal criterion RC;

k) repeating steps b) to j) until a predetermined stop criterion STC is reached; and

l) outputting the subset Su of molecules according to step k).

Said recombination scheme may comprise weighted vector additions of the descriptors of each couple of molecules MX, MY, whereby the sum of the respective weights is equal to unity.

According to an embodiment of the invention, said predetermined number of descendant molecules of step e) is two, and the weights for said vectorial additions are p and (1-p) for producing the first descendant, and 1−p and p for producing the second descendant, whereby 0≦p≦1.

Further, each descendant molecule MO which is not contained in said set S of molecules may be replaced by the one molecule of said set having the smallest distance to said descendant molecule, said distance being calculated according to a predetermined metric criterion MC.

Said recombination scheme may comprise combining a predetermined number of vector elements from the first descriptor X with a predetermined number of vector elements from the second descriptor Y.

Further, if a modified descendant does not correspond to a molecule comprised in the set S of molecules, the fitness of said descendant molecule may be calculated by using the descriptor X of the molecule of said set S having the smallest distance to said modified descendant descriptor, to a predetermined descriptor according to a predetermined metric criterion MC.

The method according to claim 1, wherein said metric criterion MC may be defined by:

$$MC ( X , Y ) = \sqrt{{\sum }_{i = 1}^{n} {( {x}_{i} - {y}_{i} )}^{2}}$$

with

xᵢ: vector element of said first descriptor X,

yᵢ: vector element of said second descriptor Y,

n: number of vector elements of said first and second descriptor, respectively.

Said selection criterion SC may comprise the Roulette Wheel type wherein the probability q of selection of a molecule M is related to its fitness f(M).

Said fitness values f of said descriptors X may be scaled by

scal(f(X))=a·f(X)+b

with a, b being constants.

Said mutation scheme may be defined by addition of a random value r_{Φ} to each said vector element x₁, said random value characterized by a probability density distribution Φ with 0 expectancy and a predetermined value for the standard deviation,

X₁^{Mut}=X₁+r_{Φ}.

The probability density distribution Φ (may be a Gaussian distribution.

The stop criterion STC may be defined by a predetermined number of repetitions of said steps b) to j), or by a predetermined limit of change in fitness.

The invention may comprise additionally also a step of visualizing the outputted molecules.

The use of a genetic algorithm is particularly advantageous due to the good adaptability to changes in descriptor length. The genetic algorithm is very robust and has excellent convergence. Furthermore, the kind of fitness function can be freely chosen, there is no need for continuity or differentiability of the fitness function.

According to the invention, only a very small amount of molecules within the data set have to be really calculated. This results in a considerable gain of performance. The iterative proceeding allows to study the data base based on customizable quality criteria. Stop and quality criteria for the search can be tailored according to a given problem.

Thus, as examples have shown, active molecules can be identified from data sets by explicitly calculating/measuring just 4-6% of the molecules within the set of molecules.

By using the method according to the invention, drug lead candidates can be identified without the need of making large molecule sets physically available and testing them. The outputted molecules are suitable for chemical synthesis.

Preferably, the molecular properties represented by said descriptors are at least two of:

molecular weight,

number of rotatable bonds,

number of hydrophobic groups,

number of hydrophilic groups,

number of acid groups,

number of basic groups,

number of neutral groups,

number of zwitter groups,

number of heavy atoms,

number of H-bond donors,

number of H-bond acceptors,

number of 1-2 dipoles,

number of 1-3 dipoles,

number of 1-4 dipoles.

The invention comprises also a computer system having means for performing the identifying method, means for inputting commands to the system, and means for outputting the result of performing the method.

Said set of molecules may be held advantageously in a computerized database.

The invention comprises also data storage means storing a program for performing the inventive method.

Further, the invention comprises data storage means storing a database comprising the set of molecules for use with the inventive method, as well as a database to be used with the inventive method.

The inventive method comprises also further a final step of testing said found candidate molecules in a suitable biological assay.

The invention and examples thereof are described in detail with reference to the accompanying figures, in which

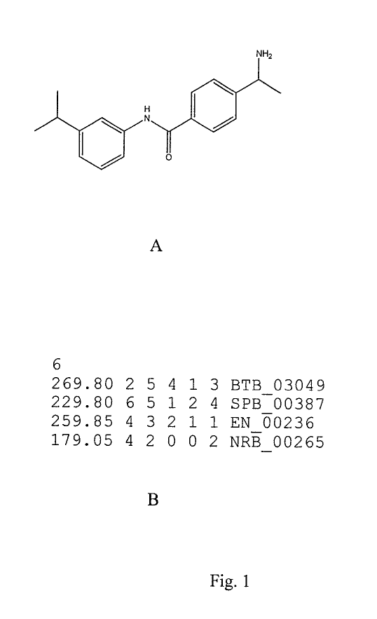

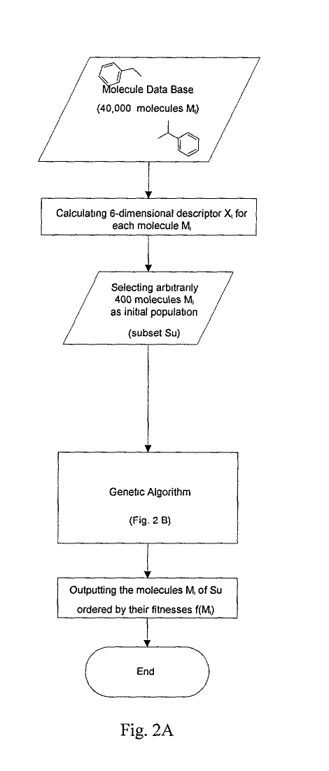

FIG. 1 a 2-D structure of a molecule, and illustrates the type of descriptor used herein,

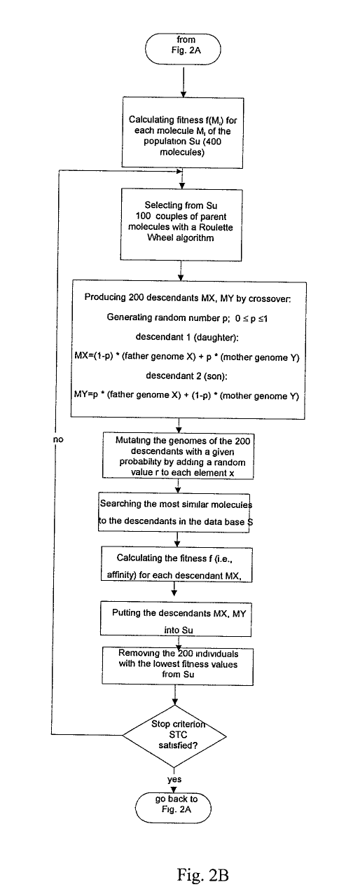

FIGS. 2A, B illustrate an embodiment of the inventive method,

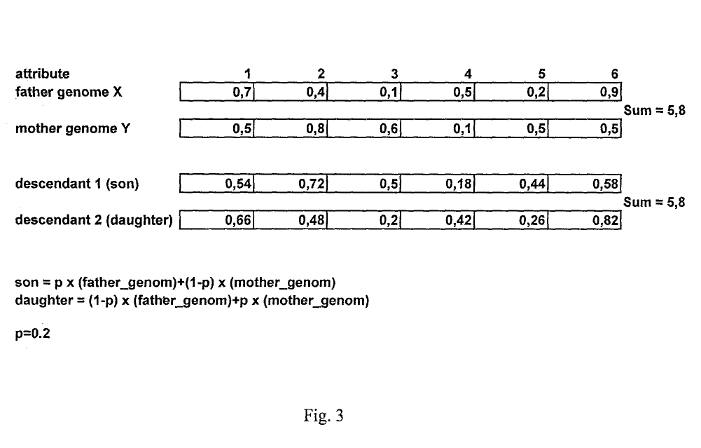

FIG. 3 illustrates a cross-over scheme,

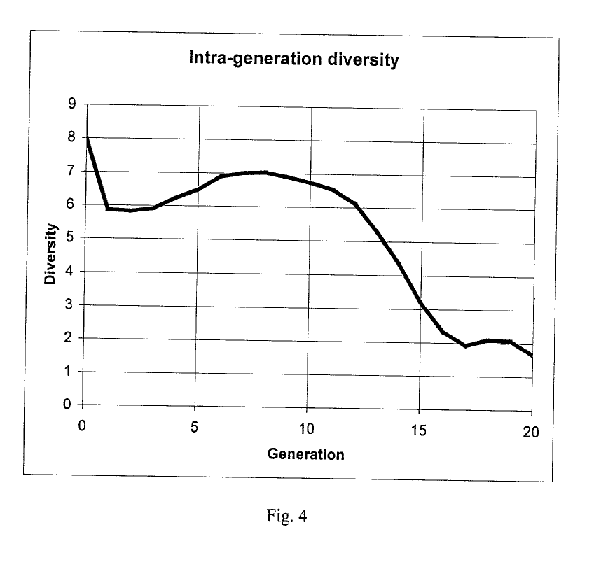

FIG. 4 illustrates the intra-generation diversity,

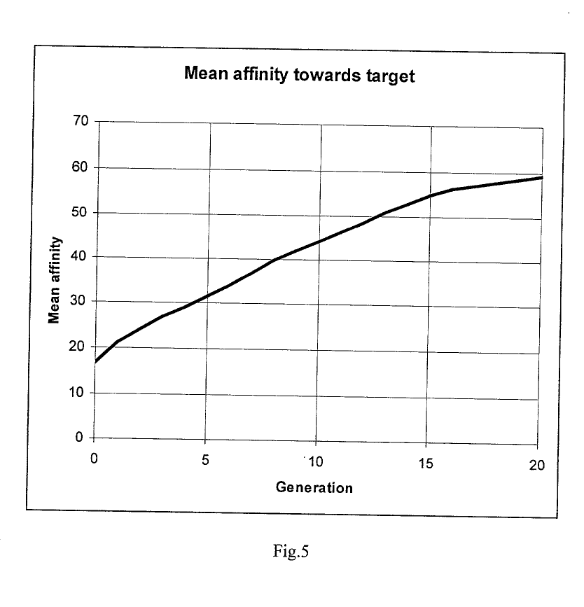

FIG. 5 illustrates the mean affinity over the population towards a target,

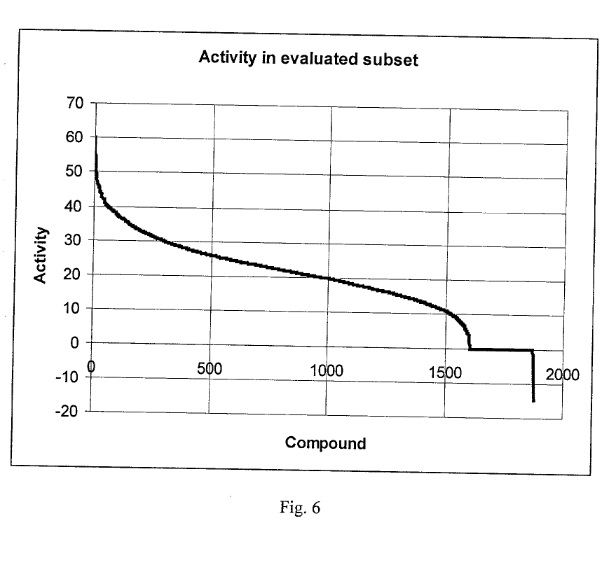

FIG. 6 illustrates the distribution of activity over the evaluated subset, and

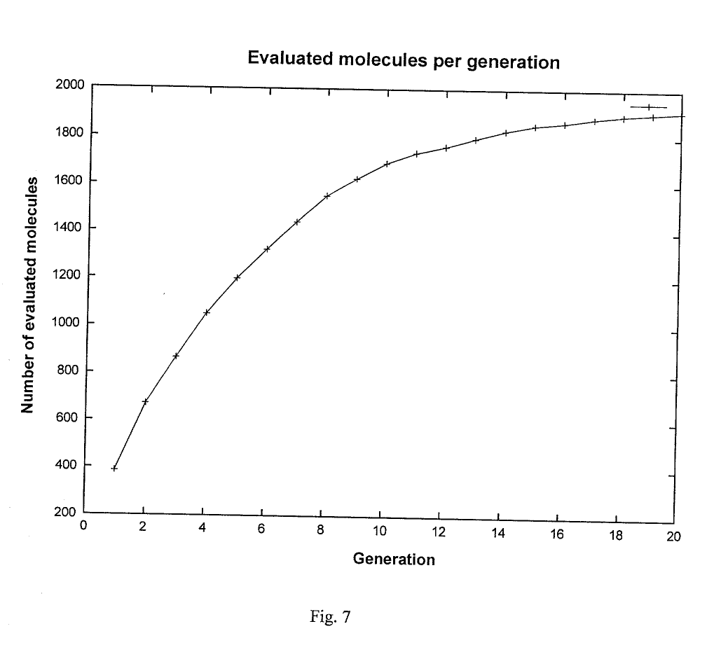

FIG. 7 illustrates the total number of evaluated molecules over the generations.

According to the invention, prior to evaluation of particular molecules, a so-called virtual library S is created, which comprises all possible molecules M. That means that the virtual molecule library contains such molecules which can be purchased or produced with reasonable costs, that are commercially available molecules or molecules which can be produced using combinatorial synthesis approaches. Not be comprised should molecules which are a priori not suitable for drug synthesis, in particular such molecules which contain toxic groups, or which have a molecular weight greater than 500 u or more than 5 donors, or molecules having a log P value of greater than 5. The library is organized as a computer database. The database in this example comprises 40,000 molecules from the World Drug Index. Each of the molecules is represented by 2-D structural data in a machine-readable form. An exemplary 2-D molecule structure is graphically shown in FIG. 1A.

Upon storing the molecules in the library, a descriptor X is assigned to each molecule M of the library, which descriptor X correlates with the biological activity of the respective molecule M. The descriptor X is a vector (x₁, . . . , xₙ) of several molecular properties, each property described by a scalar value x₁. This vector X comprises as elements x₁, . . . , xₙ the following molecular properties:

molecular weight,

number of rotatable bonds,

number of hydrophobic groups,

number of heavy atoms,

number of H-bond donors,

number of H-bond acceptors.

In order to perform a pre-selection of molecules, it is possible to use values covering economical or technical aspects, such as availability and production costs of molecules.

FIG. 1B displays, as an example, four vectors (denoting four molecules) of the descriptor used in this example. The first line specifies the dimension of the descriptor (6), the second to fifth lines specify the molecules, whereby the last element of each vector contains the ID of the corresponding molecules.

The descriptors X are adapted for further processing the molecule library S in order to find out the best molecule candidates for drug synthesis. In order to allow further processing, the descriptors chosen for the molecules of the database are all of the same dimension.

The most straightforward approach to search those molecules having the highest values of biological activity over the molecule distribution would consist in directly computing the biological activity of all the molecules of the library. However, such an exhaustive approach would be too much time consuming. Therefore, a faster search has to be performed. According to the invention, this search is performed by applying a Genetic Algorithm (GA).

FIG. 2A, B show the steps of an embodiment of the inventive method including the GA.

In the frame of a GA, the descriptor X of a molecule M corresponds to the genome of the respective individual (the molecule M).

In the first step, a subset Su comprising 400 molecules is selected from the set of molecules, whereby the selection is arbitrary. The selection may be performed by applying a Roulette Wheel algorithm. A type of Roulette Wheel algorithm will be described later. The selected subset Su is the initial population.

For each of the molecules M of the subset Su, the respective affinity f(M) to a target is computed on the basis of the molecule descriptor X. The affinity is called fitness of the individual (i.e., the molecule). The fitness f(M) may be computed by use of a docking program. For computation of the fitness, reference is made to: B. Kramer, M. Rarey, and T. Lengauer: “Evaluation of the FlexX incremental construction algorithm for protein-ligand docking PROTEINS: Structure, Functions, and Genetics”, Vol. 37, pp. 228-241, 1999, or T. Lengauer and M. Rarey: “Computational Methods for Biomolecular Docking Current Opinion in Structural Biology”, Vol. 6, pp. 402-406, 1996.

In the recombination step, the 400 molecules are the basis for producing 200 descendant individuals by recombining their respective descriptors (genomes). The recombination are crossover and mutation steps. Depending upon the way of recombining, the produced descendants, each being identified by a descriptor X, may not necessarily match with “real” molecules M comprised in the set S of molecules. Therefore, to each descendant which has no match with a real molecule of the dataset S, the one molecule of the dataset having the smallest distance to the respective descendant is determined (the “most similar” real molecule). As a measure for the distance between such an descendant and a molecule, the Euclidean distance MC of the respective descriptors X, Y is used,

$$MC ( X , Y ) = \vert X , Y \vert = \sqrt{{\sum }_{i = 1}^{n} {( {x}_{i} - {y}_{i} )}^{2}} ,$$

wherein x₁ denotes a vector element of the first descriptor X, and y, denotes a vector element of the second descriptor Y. It should be noted that other metrics may be applied, e.g. Cosinus-Coefficient, Tanimoto-Coefficient, Mahalanobis-Distance.

The degree of crossover and mutation is determined by specific parameters found by empirical methods. Crossover and mutation will be described later.

Then, for each of the 200 descendant molecules (MO), the respective fitness f(MO) is computed. It has been found out that for a good convergence of the GA it is preferable to compute the fitness on the basis of the descriptors of real molecules M found in the population (i.e., the most similar molecules) rather than on the basis of the exact descriptors X of the descendants as obtained by the reproduction process.

The descendants MO (described by their respective exact descriptors XO) are part of the subset Su of molecules, thus containing 600 individuals. From these 600 individuals, the worst 200 molecules are removed in order to keep the number of individuals of the subset Su constant.

On the basis of the so remaining subset (population) of 400 individuals, in the next epoch 200 new individuals are produced, in the same way as described above. The procedure is repeated for 20 epochs (i.e., iterations). Alternatively, the number of epochs (iterations) may be determined by evaluating a stop criterion after each iteration. As stop criterion, the changes in fitness of the produced individuals may be used. If the sum of the changes of the respective fitness values between two generations decreases below a given threshold, the process can be stopped.

The recombination step for producing the new individuals is performed in the following way: From the subset of 400 molecules, 100 (parent) couples of molecules (MX, MY) are chosen. The selection is performed by a random selection over the 400 individuals, whereby the probability of an individual of being selected is directly proportional to its fitness. The larger the fitness, the larger the selection probability of the respective individual. The algorithm used is of the “Roulette wheel” type, which will be described later.

The crossover is performed by weighted vectorial addition of the descriptors X, Y of the parent couple, whereby weights p are defined as a weighting factor. The factor p is determined for each couple separately as a random number between 0 and 1.

FIG. 3 gives an example of the crossover step for two parent genomes, whereby the weighting factor is p=0.2. The respective elements x₁, y₁ of the descriptors X, Y are added, whereby the elements of one descriptor X are weighted with the weighting factor p, and the elements of other descriptor Y are weighted with the complement to 1, i.e., with (1−p).

One of the crucial points of the GA is the evaluation of the fitness values. Since the distribution of the fitnesses f(X) of the individuals may not be equilibrated over a given range, the fitness values f(X) are scaled to a predetermined range prior to performing the “Roulette Wheel” removal step. As formula for scaling the fitnesses f(X), the following “stretching” relation has been found to be suitable: scal(f(X))=a·f(X)+b, with a, b being constants. After scaling the fitness values, the worst 200 molecules are removed from the subset Su.

The mutation is characterized by addition of a random value r_{Φ} characterized by a probability density distribution Φ with 0 expectancy and a predetermined value for the standard deviation, x₁^{Mut}=x₁±r_{Φ}. The probability density distribution Φ of the random value r_{Φ} can be, for example, a Gaussian function.

If a resulting vector element value xᵢ is out of the given range (xₘᵢₙ, . . . , xₘₐₓ), the vector element x₁ may be corrected in the following way:

x₁^{Mut}=x₁±r_{Φ}mod max(x₁),

wherein max(x₁) denotes the maximum value of the element xᵢ, over the set of molecules. The plus (+) sign is applied if the vector element value is inferior to the lower border xₘᵢₙ of the range, the minus (−) sign is applied if the vector element value is greater than the upper border xₘₐₓ of the range.

The performance of the method according to the invention was evaluated with a 40,000 molecule Set of the World Drug Index. The inhibition of the enzyme scd1 was measured in terms of target-receptor-affinity. The genetic algorithm was ran over 20 generations. As the algorithm proceeds, the intra-generation diversity is decreased during the iterations as shown in the FIG. 4.

During the same process, the mean affinity towards the target is increased, as shown by FIG. 5. That means that the subsequent population yields better individuals. As a result of the whole process, a 1877 molecule subset of the database is evaluated, resulting in a strong enrichment of pharmacological active compounds.

FIG. 6 illustrates the distribution of activity over the evaluated subset. 944 of the molecules show at least slight activity, 300 show strong activity, 59 of the compounds can be considered as potential lead structures for further development with activities greater than 40 kJ/mol. The most active compound included in the subset (60 kJ/mol) was found.

FIG. 7 displays the total (cumulative) number of evaluated molecules over the generations (i.e., including molecules evaluated in earlier generations).

The time needed for the evaluation of the subset was 3756 minutes (2 min per molecule, 2 min for the algorithm), whereby the GA algorithm was implemented in C++ and was run on a 400 MHz computer system. The data base was based on a Oracle 8.15 RDBMS.

The identified molecules may be tested in suitable biological assays as described for instance by R. Bolger, “High-throughput screening: new frontiers for the 21^{st} century”, published in DDT, Vol. 4, No 6, pp. 251-253, June 1999, or by J. S. Major, “Challenges of high throughput screening against cell surface receptors”, J. of Receptor and Signal Transduction Research, 15(1-4), pp. 595-607, 1995.

1. A method of identifying biologically active molecules from a set (S) comprising a predetermined number (N) of different molecules (M1, M2, . . . , MN), said molecules being expected to be biologically active with respect to a predetermined target (T), each said molecule (M1, M2, . . . , MN) of said set (S) being identified by a machine-readable descriptor (X1, X2, . . . , XN), respectively, each said descriptor (X1, . . . , XN) being a vector with n vector elements (x1, . . . , xn), n being a natural number, each vector element (x1, . . . , xn) representing a predetermined molecular property, said method comprising the following steps:
a) selecting arbitrarily from said set (S) of molecules a subset (Su) comprising a predetermined first number (Nu) of molecules (Mi, . . . , Mk);
c) assigning a fitness (fi, . . . , fk), to each molecule (Mi, . . . , Mk) of said subset (Su), respectively, said fitness (fi, . . . , fk) being calculated according to a predetermined fitness measure f(X), said fitness measure f(X) being representative of the affinity of a molecule (Mi, . . . , Mk) to said target (T);
m) establishing, according to a predetermined selection criterion (SC), from said subset (Su) a predetermined number (nc) of couples of molecules (MX, MY);
n) with each established couple of molecules: producing a predetermined number of descendant molecules (MO1, MO2) by recombining the descriptors (X, Y) of said couple of molecules (MX, MY) according to a predetermined recombination scheme;
o) mutating each said descendant molecule (XM) by modifying the respective descriptor (XO) according to a predetermined mutation scheme (MS);
p) assigning a fitness (f) to each modified descendant molecule (MO), said fitness f(MO) being calculated according to the fitness measure f(X) of step b);
q) adding said modified molecules (MO) to said subset (Su);
r) removing a predetermined number of molecules from said subset (Su), the molecules to be removed being determined by a predetermined removal criterion (RC);
s) repeating steps b) to j) until a predetermined stop criterion (STC) is reached; and
t) outputting the subset (Su) of molecules according to step k).

2. The method according to claim 1, wherein said recombination scheme comprises weighted vector additions of the descriptors of each couple of molecules (MX, MY), whereby the sum of the respective weights is equal to unity.

3. The method according to claim 2, wherein said predetermined number of descendant molecules of step e) is two, and the weights for said vectorial additions are p and 1-p for producing the first descendant, and 1-p and p for producing the second descendant, whereby 0≦p≦1.

4. The method according to claim 1, wherein each descendant molecule (MO) which is not contained in said set (S) of molecules is replaced by the one molecule of said set having the smallest distance to said descendant molecule, said distance being calculated according to a predetermined metric criterion (MC).

5. The method according to claim 1, wherein said recombination scheme comprises combining a predetermined number of vector elements from the first descriptor (X) with a predetermined number of vector elements from the second descriptor (Y).

6. The method according to claim 1, wherein, if a modified descendant does not correspond to a molecule comprised in the set (S) of molecules, the fitness of said descendant molecule is calculated by using the descriptor (X) of the molecule of said set (S) having the smallest distance to said modified descendant descriptor, to a predetermined descriptor according to a predetermined metric criterion (MC).

7. The method according to claim 1, wherein said metric criterion MC is defined by:
$$MC ( X , Y ) = \sqrt{{\sum }_{i = 1}^{n} {( {x}_{i} - {y}_{i} )}^{2}}$$
with
x₁: vector element of said first descriptor X,
y₁: vector element of said second sit descriptor Y,
n: number of vector elements of said first and second descriptor, respectively.

8. The method according to claim 1, wherein said selection criterion (SC) is of the Roulette Wheel type wherein the probability (q) of selection of a molecule (M) is related to its fitness f(M).

9. The method according to claim 1, wherein said fitness values f of said descriptors (X) are scaled by
scal(f(X))=a·f(X)+b
with a, b being constants.

10. The method according to claim 1, wherein said mutation scheme is defined by addition of a random value rΦ to each said vector element (xᵢ), said random value characterized by a probability density distribution (Φ) with 0 expectancy and a predetermined value for the standard deviation,
xᵢ^{Mut}=xᵢ+r_{Φ}.

11. The method according to the preceding claim, wherein probability density distribution (Φ) is a Gaussian distribution.

12. The method according to claim 1, wherein said stop criterion (STC) is defined by a predetermined number of repetitions of said steps b) to j).

13. The method according to claim 1, wherein said stop criterion (STC) is defined by a predetermined limit of change in fitness.

14. The method according to claim 1, comprising a step of visualizing the outputted molecules.

15. The method according to claim 1, wherein said set of molecules is held in a computerized database.

16. The method according to claim 1, comprising a step of visualizing the resulting 3-D surfaces.

17. The method according to claim 1, wherein said selected candidate molecules are suitable for chemical synthesis.

18. The method according to claim 1, whereby the molecular properties represented by said descriptors are at least two of:
molecular weight,
number of rotatable bonds,
number of hydrophobic groups,
number of hydrophilic groups,
number of acid groups,
number of basic groups,
number of neutral groups,
number of zwitter groups,
number of heavy atoms,
number of H-bond donors,
number of H-bond acceptors,
number of 1-2 dipoles,
number of 1-3 dipoles,
number of 1-4 dipoles.

19. The method according to claim 1, whereby the molecular properties represented by said descriptors are:
molecular weight,
number of rotatable bonds,
number of hydrophobic groups,
number of heavy atoms,
number of H-bond donors,
number of H-bond acceptors.

20. The method according to claim 1, whereby the molecular properties represented by said descriptors are at least two of:
molecular weight,
number of rotatable bonds,
number of hydrophobic groups,
number of heavy atoms,
number of H-bond donors,
number of H-bond acceptors.

21. A computer system comprising means for performing the method according to claim 1.

22. The computer system according to the preceding claim comprising means for communicating with a database comprising said set of molecules.

23. A data storage means storing a program for performing the method according to claim 1.

24. A data storage means storing a database comprising the set of molecules for use with the method according to claim 1.

25. A program for storing a database comprising the set of molecules for use with the method according to claim 1.

26. A database to be used with the method according to claim 1.

27. Method of producing molecules determined by the method according to claim 1.

28. Method according to claim 27, further comprising a final step of testing said found candidate molecules in a suitable biological assay.
